(19) United States
(12) Patent Application Publication
(10) Pub. No.: US 2021/0393407 A1
(43) Pub. Date: Dec. 23, 2021

(54) BIOSCAFFOLD FOR IN VIVO USE

(71) Applicant: The Board of Supervisors of Louisiana State University and Agricultural and Mechanical College, Baton Rouge, LA (US)

(72) Inventor: Giovanni SOLITRO, Shreveport, LA (US)

(21) Appl. No.: 17/281,889

(22) PCT Filed: Oct. 1, 2019
(86) PCT No.: PCT/US2019/054101

§ 371 Date: Mar. 31, 2021

Related U.S. Application Data
(60) Provisional application No. 62/739,488, filed on Oct. 1, 2018.

(51) Int. Cl. A61F 2/28; A61L 27/38

(52) U.S. Cl. CPC A61F 2/2846; A61L 27/3821; A61L 27/3808; A61F 2002/30677; A61L 27/3834; A61L 27/3813; A61L 27/3847; A61L 27/383

(57) ABSTRACT

This invention is directed to structural units and bioscaffolds that comprise the same for in vivo use.

This application claims priority from U.S. Provisional Application No. 62/739,488, filed on Oct. 1, 2018, the entire contents of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to structural units and bioscaffolds that comprise the same for in vivo use.

### BACKGROUND OF THE INVENTION

In presence of large bone defects, a scaffold serves as a matrix for the regeneration of tissue. In current approaches, biocompatibility, degradability, and cell adhesion are given to the scaffolds through the materials composing the scaffold using synthesis of a bioceramic materials such as hydroxyapatite or synthetic polymers such as PLA. Cell hosting and nutrition are conferred through porosity and scaffold designs. Existing scaffolds are mostly created by imitating bone architecture (FIG. 1), at the point that the scaffold is obtained through segmentation of microCT data. The trabeculae are modelled as beams and cell and nutrient hosting is required to the obtained trusses which are characterized by small adhesion surface and high porosity. Compared to actual bone, nutrient transport and mechanical stimuli are not locally regulated.

### SUMMARY OF THE INVENTION

Aspects of the invention are directed towards a structural unit comprising, a central pillar and a plurality of load arms.

In embodiments, a load arm of the plurality of load arms comprises a bending arm and a rib.

In embodiments, the central pillar and the plurality of load arms are configured to receive at least one force.

In embodiments, the bending arm secures the rib to the central pillar.

In embodiments, the rib follows a curved pathway transitioning around an elbow to a return pathway in the direction of the central pillar. In other embodiments, the rib follows a straight pathway. In embodiments, the curved pathway can comprise variable angles and/or multiple angles. For example, the curved pathway is concave downward relative to the transverse plane, concave upward relative to the transverse plane, or a combination thereof. In embodiments, the curved pathway comprises a constant radius of curvature, a variable radius of curvature, or a combination thereof.

In embodiments, the central pillar can be solid or hollow.

In embodiments, the structural unit further comprises a pivot arm, wherein the pivot arm secures the rib to the central pillar.

In embodiments, the plurality of load arms are integrally formed.

In embodiments, one or more axial lines and one or more transverse planes orient the structural unit. The axial line can be collinear with a first axis of the central pillar. The transverse plane can be perpendicular to the axial line.

In embodiments, the structure displays non-axial symmetry, such as a structure that is not axially symmetric.

In embodiments, the plurality of load arms can be oriented in perpendicular relation to the transverse plane. In embodiments, the plurality of load arms can be oriented about 10°, about 20°, about 30°, about 40°, about 50°, about 60°, about 70°, about 80°, about 90° relative to the transverse plane.

In embodiments, the plurality of load arms can comprise a second axis collinear with the axial line.

In embodiments, the plurality of load arms are radially spaced about the second axis. In embodiments, the plurality of arms are viably spaced. In embodiments, the plurality of arms are equally spaced, unequally spaced, or any combination thereof.

In embodiments, the at least one force comprises a vector collinear with the axial line and/or a vector oblique to the axial line.

In embodiments, the force comprises contact pressure.

In embodiments, the receiving the at least one force including causing a deformation of the structural unit along the transverse plane.

In embodiments, a plurality of cross sectional planes cross section the structural unit, wherein the plurality of cross sectional planes are parallel to the transverse plane.

In embodiments, each cross section comprises a cross sectional radius.

In embodiments, expansion, and stasis of cross sectional radii.

In embodiments, characteristics of the at least one of a contraction, expansion, and stasis of cross sectional radii depend upon at least one parameter of the structural unit.

In embodiments, the at least one parameter comprises radial spacing between ribs of the plurality of ribs, thickness of the plurality of ribs, angular width of the plurality of ribs, length of the at least one bending arm, length of the at least one pivot arm, positioning of the at least one pivot arm, or any combination thereof.

In embodiments, the transverse plane separates a first set of load arms in a first region and a second set of load arms in a second region, wherein the at least one parameter comprises at least one gap between pivot arms in the first region and pivot arms in the second region, wherein the plurality of load arms comprise the first set of load arms and the second set of load arms.

In embodiments, the structural unit comprises a sphere, a conical shape, an oval shape, and a cubical shape.

In embodiments, the sphere comprises a diameter of approximately 50 μm to 2000 μm.

In embodiments, the structural unit comprises a symmetric shape or an asymmetric shape.

In embodiments, the structural unit comprises a biocompatible material.

In embodiments, the biocompatible material comprises a photoresist polymer or a polymer that is compatible with three-dimensional printing technology, such as (2-(Hydroxymethyl)-2-[[(1-oxoallyl)oxy]methyl]-1,3-propanediyldiacrylate, known as IP-L; Photoresist pentaerythritol tetraacrylate (PETTA, Sigma-Aldrich) containing 3% Irgacure 379 photoinitiator (Ciba); polyurethanes polycaprolactone, polyglycolic acid, polylactic acid, polyamides, polyolefin, polyester, polytetrafluoroethylene, polyurethanes, and hydrogels used for bioprinting such as collagen, alginate, agarose, and chitosan, and synthetic hydrogels such as hyaluronan-methylcellulose, polyethylene glycol diacrylate collagen, laminin, matrigel, and non-biodegradable materials such as polysiloxanes, Stainless steel, Co—Cr alloys, Ti-alloys.

Embodiments further comprise an external extended surface surrounding the structural unit.

In embodiments, the external extended surface provides an inner region.

In embodiments, the inner region comprises a fluid, non-limiting examples of which comprise media such as DMEM Media, DMEM/F12 Media, Ham's F-10 and F-12 Media, Medium 199, MEM, RPMI 1640 Media, Serum. In embodiments, the inner region comprises at least one bioactive agent, which can refer to virtually any substance which possesses desirable characteristics for application to the implant site

In embodiments, the structural unit further comprises the capability of hosting incompressible and compressible fluids in a scaffold without openings. Without being bound by theory, the deformation of the unit is regulated by Boyle's Laws.

In embodiments, the external extended surface is fully sealed.

In embodiments, the external extended surface comprises at least one opening, wherein the at least one opening releases one or more medications over time when implanted in vivo, establishes an equilibrium between external and internal pressures, supplies substances including nutrients, filters a stream surrounding the structural unit, and fit units together.

Embodiments can comprise a superior portion and an inferior portion.

In embodiments, the structural unit can be manufactured by coupling two pre-built portions, such as hemispheric portions.

In embodiments, the structural unit can be manufactured by assembling prebuilt components.

In embodiments, the superior portion and the inferior portion are flat.

In embodiments, the superior portion and the inferior portion are radially extended.

In embodiments, connecting elements connect the plurality of load arms to the central pillar. Connecting elements can be manufactured from a material identical to or stiffer than that of the core structural unit. Such materials comprise metallic materials.

Aspects of the invention are also directed towards a composition comprising two or more structural unites of claim 1 and a pharmaceutically acceptable carrier.

In embodiments, the two or more structural units can suspended or randomly dispersed in a media, such as a fluid, spray, solid, semi-solid, gel, or powder, to provide a composition. In embodiments, the media is a pharmaceutically acceptable media, such as a pharmaceutically acceptable carrier. In embodiments, the media comprises matrigels and/or hydrogels. As such, the composition is suitable to be administered to a subject. In embodiments, the structural units can be embedded in an object, such as a bone screw, prosthesis or corresponding coating. In embodiments, the fluid comprises agar.

In embodiments, the structural unit can comprise at least one bioactive agent, which can refer to virtually any substance which possesses desirable characteristics for application to the implant site

Still further, aspects of the invention are directed towards a bioscaffold of at least two structural units described herein, wherein the at least two structural units are one or more of indirectly connected using connecting elements and directly connected.

In embodiments, the bioscaffold comprises a sheet, a stack, a spiral, or a linear bioscaffold.

In embodiments, the at least two structural units are at least one of identical, similar, and different.

In embodiments, at least one of the at least two structural units comprises memory alloy, an electric motor, reacts to external magnetic fields, expands or contracts based on changes in environment (such as body temperature and/or body loads) or any combination thereof.

In embodiments, the bioscaffold at least one of regulates fluid flow through the bioscaffold, regulates nutrient flow through the bioscaffold, provides mechanical stimuli, and promotes proliferation and survival of cells.

In embodiments, the bioscaffold comprises viable cells.

In embodiments, the cells comprise exogenous cells, autologous cells, and allogenic cells.

In embodiments, the viable cells comprise one or more of osteoblasts, osteaoclasts, lining cells, stromal cells, fibroblasts, endothelial cells, progenitor cells, stem cells, organ-specific cells, tissue-specific cells, keratinocytes, melanocytes, and a nerve cell.

Yet further, aspects of the invention are directed towards a method of regenerating tissue in a subject, the method comprising obtaining a bioscaffold described herein and implanting the bioscaffold to a site on the subject, whereby the bioscaffold is populated with viable cells, thereby regenerating a tissue in a subject.

In embodiments, the tissue comprises bone.

In embodiments, the bioscaffold is provided on a prosthesis or implant, or as a coating or film, such as to cover bone screws.

Other objects and advantages of this invention will become readily apparent from the ensuing description.

### BRIEF DESCRIPTION OF THE FIGURES

The present disclosure is focused on structural units and bioscaffolds that comprise the same for in vivo use. It is to be understood that this disclosure is not limited to particular embodiments described, and as such may, of course, vary. Further aspects of the present disclosure will be readily appreciated upon review of the detailed description of its various embodiments, described below, when taken in conjunction with the accompanying drawings.

The patent or application file contains at least one drawing executed in color. Copies of this patent or patent application publication with color drawing(s) will be provided by the Office upon request and payment of necessary fee.

FIG. 1 shows the truss scaffolds.

FIG. 2 shows auxetic structural unit shown in (a) transparence and (b) isolated internal structure.

FIG. 3 shows draft of a structure realized with 3D printing.

FIG. 4 shows examples of units profiles (continues line) and their deformation patterns (dashed lines) that could be obtained by modulating rib dimensions and position of the connecting arms.

FIG. 5 shows auxetic structural unit organized in matrix 3×3×2 in transparence and with isolated internal structure shown as (a) CAD model and (b) manufactured in diameter of 100 μm.

FIG. 6 shows half unit showing an extended circular base and connecting elements in two different shapes to ease the 3D printing.

FIG. 7 shows examples of units organized to compose layers of 2D or 3D tridimensional scaffolds.

FIG. 8 shows examples of units connected in different manners to compose scaffolds shown from the top.

FIG. 9 shows an example on how volumes of fluid are modulated during scaffold deformation coupling units with incompressible behavior in green with units that exhibits a negative Poisson's ratio in blue followed by an example of organized matrix with pre-established nutrients path.

FIG. 10 shows example of auxetic scaffold generated for femoral neck fracture: (a) location in antero-posterior view, (b) scaffold, and (c) its isometric view.

FIG. 11 shows block diagram that could be included in an algorithm developed to reconstruct scaffolds from diagnostic imaging.

FIG. 12 shows an embodiment of the invention.

FIG. 13 shows an illustration of cross section variability within a single structural unit.

FIG. 14 shows an illustration of an asymmetric embodiment of the invention.

FIG. 15 shows an illustration of variable radial disposition of the loading arms. For example, each arm can be developed with variable curvature and sections. The variability of the section can be given in terms of dimensions and shape, for example. The number and dimensions of the curvatures and sections can be variable depending on the function use of the structural unit.

FIG. 16 shows an illustration of a scaffold with no ribs.

FIG. 17 shows an illustration of a unit and an external shell. (a) The unit can be totally covered by an external shell, or (b, c) the unit can be partially covered by an external shell. Non-limiting examples of configurations with partial coverage are those wherein there is an opening (b) in correspondence of the transverse middle place or (c) in correspondence with the two apexes.

FIG. 18 shows an illustration of a unit and an external shell. The external shell can be continuous when surrounding one or more units. For example, the external shell can be fabricated with an elongated profile that works as a sleeve for one or more units.

FIG. 19 shows an illustration of a unit and an external shell. The external shell can also be characterized by (a) constant thickness, (b) a thickness that varies in relation to the polar coordinates, or (c) can have localized variation of thickness (such as thinning or thickening) to exhibit localized deformations or to guarantee durability of the structure in the points mainly subject to stress. The examples illustrated herein show examples of variations of thickness along the transverse plane, but it will be understood that the variations in thickness can also be created for different planes and can also be create in non-uniform patterns.

FIG. 20 shows that the external shell can be (a) articulated and have a profile recessed in the unit, (b) directly connected to the central pillar with external continuity, (c) directly connected to the central pillar without external continuity, or (d, e) have complex connections to the ribs to accommodate compression without stress concentrations or to fine modulate its deformation pattern.

FIG. 21 shows that the external shell (a) can be formed by multiple materials, (b) can be composed of different layers that can be concentric, (c) can be composed of different layers that are partially distributed, (d) can comprise holes, or € can be limited to certain portions. For example, such variations can be used to modulate its biodegradability, to stiffen certain regions, or simply for ease of manufacturing.

FIG. 22 shows that the external shell can also be composed of independent sections attached to the ribs that can be (a) disjoined or (b) partially superimposed (or overlapping) like in a rose. For example, such variations can be used for easy deformation of the unit while adopting a rigid material.

FIG. 23 shows that portions of the unit can comprise one or more support structures. For example, the superior and inferior portions of the unit can be enriched by one or more supports that, for example, can be used to stabilize the unit within the scaffold, to increases the torque on the ribs or simply to link multiple ribs that can be actuated simultaneously. It will be understood that these supports can also be of different heights within a single unit. As illustrated, the supports can be made with (a) a flat surface, (b) rounded edges, (c) describe a circular or elliptical surface, (d) able to enclose a cylinder, or (e) simply flat in relation to the desired connection. Must be noted that properly profile these supports it is possible to influence the loading of the unit.

FIG. 24 shows that multiple units can be organized in several manners. For example, multiple units can be (a) in simple contact, (b) bonded, (c) for interposition of a third element, (d) or through the supports.

FIG. 25 shows the organization of multiple units. For example, the “supports” can be of (a) simple contact between the units or (b) can be bonded to constitute a continuum. When there is a third element interposed, the shape of this element can determine the modality of deformation of the unit, (c) following an elliptical path for both units, (d) following an elliptical path on one unit while the other is actuated only after the load needed to close the gap is achieved. Note that the interpose element and also the units are not necessarily axisymmetric despite the drawings.

FIG. 26 shows that the unit can be disposed with several patterns. For example, their disposition can be dictated by the desired bone profile and their dimension can be varied within a structure in relation to the desired bone density. Both profile and density, in addition to be arbitrarily chosen can be taken from existing CT images, can be obtained as result of stress or fluid dynamic analysis, or simply dictated by limitations of the manufacturing process. Examples of scaffolds dispositions can be drawn with patterns organized as (a) a matrix, (b) honeycomb, (c) in layers of different unit dimensions disposed in planes, (d) concentric, or (e) simply composed by various shaped units not organized following an organized disposition or progression in their dimension.

FIG. 27 shows photographs of a prototype of an embodiment of the invention that was manufactured by 3D printing. Panels a-d demonstrate compression of the structural unit.

FIG. 28 shows two views of the configuration in which the ribs are extended below the mid transverse plane.

FIG. 29 shows an embodiment of the invention wherein the ribs are connected to the central pillar through pivot joints.

FIG. 30 shows images of the structural unit printed in diameters of 50 μm and 25 mm.

FIG. 31 shows an image of an embodiment of the invention.

FIG. 32 shows an image of an embodiment of the invention.

FIG. 33 shows an image of an embodiment of the invention.

### DETAILED DESCRIPTION OF THE INVENTION

### Abbreviations and Definitions

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Structural Unit

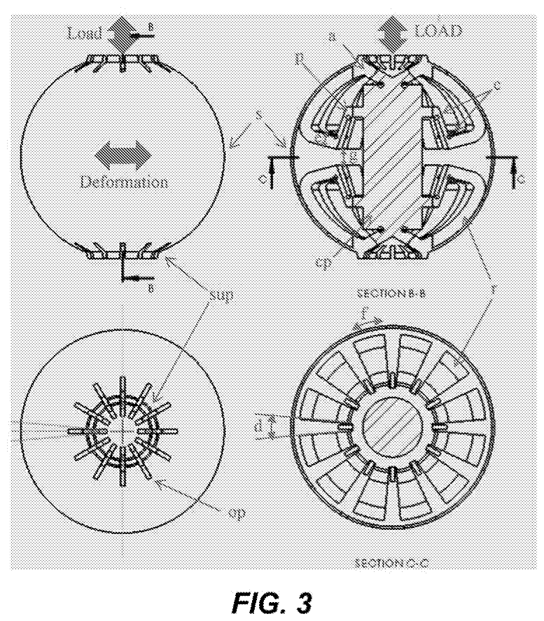

Aspects of the invention are directed towards a structural unit, compositions comprising one or more structural units, and scaffolds, such as bioscaffolds, comprising two or more structural units. FIG. 3 shows one embodiment of the structural unit. The structural unit core comprises a central pillar (“cp)” and a plurality of load arms. In embodiments, the central pillar and the plurality of load arms are configured to receive at least one force or load.

In embodiments, the central pillar can be a single monolithic body (for example a solid body or a hollow body). In other embodiments, the central pillar can be composed of two or more bodies bundled to provide a central pillar.

In embodiments, the plurality of load arms comprise at least one bending arm (“a”), at least one rib (“c”), at least one pivot arm (“p”), or any combination thereof. The bending arm can secure the rib to the central pillar. The rib can follow a curved pathway transitioning around an elbow to a return pathway in the direction of the central pillar. For example, the curved pathway can comprise a constant radius of curvature or a variable radius of curvature. The pivot arm secures the rib to the central pillar.

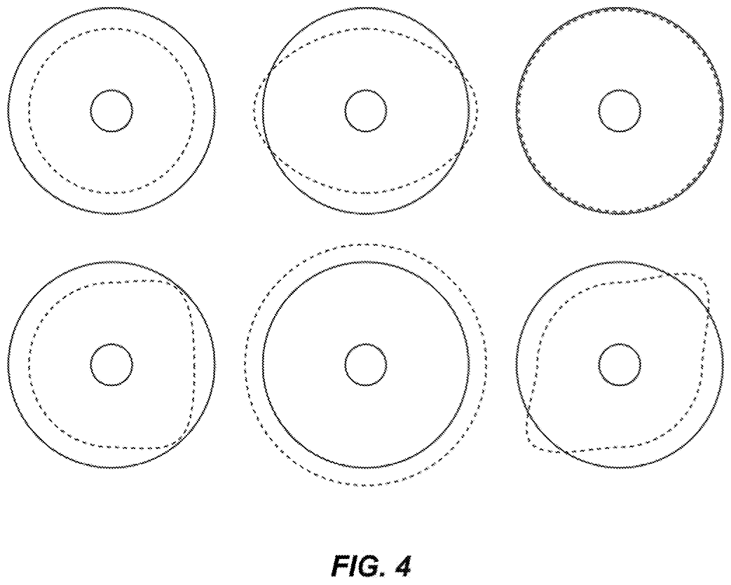

In embodiments, ribs (r) under compression form the presence of bending arms (a) which folds towards the center dragging the external shell(s). Variability in rib thickness can be used to limit deformation to specific regions of the ribs (c). The deformation of the ribs can be controlled by their geometrical dimensions, for instance thickness (e), angular width (f), length of the bending arm (a), and position of the pivoting arm (p). For example, the ribs can be equally spaced (d) and of constant dimensions in order to confer a transverse isotropic behavior to the unit. Alternatively, the ribs can be unequally spaced with eventually unequal dimensions to conferee variable transverse behaviors and stiffness with reference to specific directions (see FIG. 4, for example).

Ribs extensions can be adjusted in order to reduce the gap (g) and control the non-linear behavior in response to axial deformation.

In embodiments, the structural unit comprising ribs can be a monolithic object. In other embodiments, the structural unit can be provided as a combination of discrete elements/components. For example, the discrete elements can be produced individually, and the structural unit can then be constructed therefrom. For example, the structural unit can be obtained as combination of multiple elements that do not necessarily deform during the compression or extension of the unit. Referring to FIG. 29, for example, this is the case of ribs connected to the central pillar through pivot joints. As another example, the elastic and damping action obtained by the monolithic body is exercised by one or more discrete elements with elastic and damping capabilities.

While its Poisson's ratio is determined by the geometrical configuration conferred to the radial branches, following an initial deformation, stiffness to axial load can be regulated through the dimensions of the central pillar (cp).

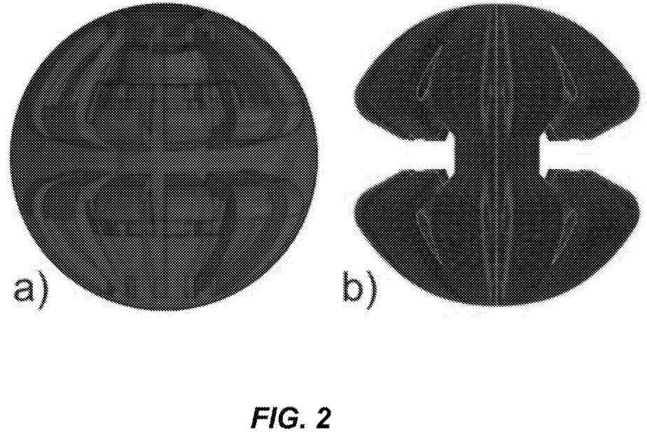

An axial line and a transverse plane can orient the structural unit, wherein the axial line is collinear with a first axis of the central pillar. In embodiments, the transverse plane is perpendicular to the axial line. Referring to FIG. 2, for example, the transverse plane can separate a first set of load arms in a first region and a second set of load arms in a second region.

The plurality of load arms can be integrally formed and can be oriented in perpendicular relation to the transverse plane. In embodiments, the plurality of load arms can be oriented about 10°, about 20°, about 30°, about 40°, about 50°, about 60°, about 70°, about 80°, about 90° relative to the transverse plane.

The plurality of load arms can comprise a second axis collinear with the axial line, and, in embodiment, the plurality of load arms can spaced about the second axis, such as radially spaced about the second axis. In embodiments, the plurality of arms are viably spaced. In embodiments, the plurality of arms are equally spaced, unequally spaced, or any combination thereof.

The skilled artisan will recognize that there can be variability in the profile of the one or more arms of the structure depending on the desired functional characteristics of the structure and/or scaffold. For example, the one or more arms can be provided as a variety of shapes and sizes so as to provide an optimal combination of various structural/function/mechanical properties, including durability, bioactive agent release characteristics, cell survivability, availability, cost, and the like. See, for example, U.S. Pat. No. 8,236,061, which is incorporated herein by reference in its entirety.

As described herein, the rib can follow a curved pathway transitioning around an elbow to a return pathway in the direction of the central pillar. In embodiments, the curved pathway is concave downward relative to the transverse plane. In other embodiments, the curved pathway is concave upward relative to the transverse plane.

Referring to FIG. 4, the structural unit can undergo deformation, such as deformation of the structural unit along the transverse plane, as a result of a force or load exerted onto the structural unit. The deformation can comprise at least one of a contraction, expansion, and stasis of cross sectional radii. Non-limiting examples of such deformating force or load comprises contact pressure or non-contact pressure, non-limiting examples of which comprise hydrostatic pressure and/or hydraulic pressure. The structural unit can be designed to withstand a force or load applied to any area. The force or load can be torsion, such as push torsion or pulled torsion, for example.

The characteristics of the deformation, such as the characteristics of at least one of a contraction, expansion, and stasis of cross sectional radii, can depend upon at least one parameter of the structural unit. For example, variability in rib thickness, angular width, length of bending arm, position to pivoting arm, and/or their geometrical dimensions can be used to limit deformation to specific regions of the ribs (c). As another example, stiffness to axial load can be regulated through the dimensions of the central pillar (cp). Non-limiting examples of parameters of the structural unit that influence deformation of the structural unit comprise radial spacing between ribs of the plurality of ribs; thickness of the plurality of ribs; angular width of the plurality of ribs; length of the at least one bending arm; length of the at least one pivot arm; positioning of the at least one pivot arm; length of the central pillar; stiffness of the central pillar; width or thickness of the central pillar; whether the central pillar is hollow or solid; the gap or distance between pivot arms in the first region and pivot arms in the second region.

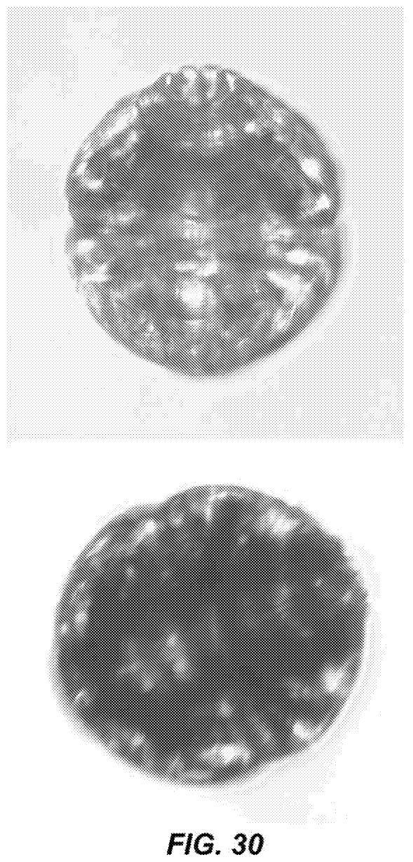

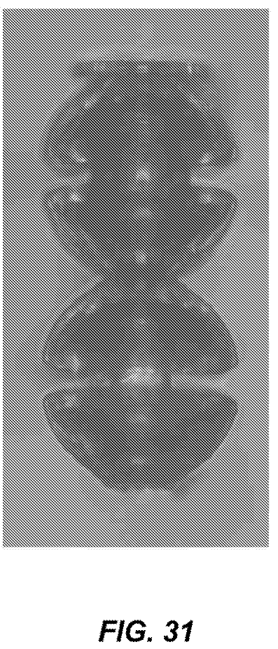

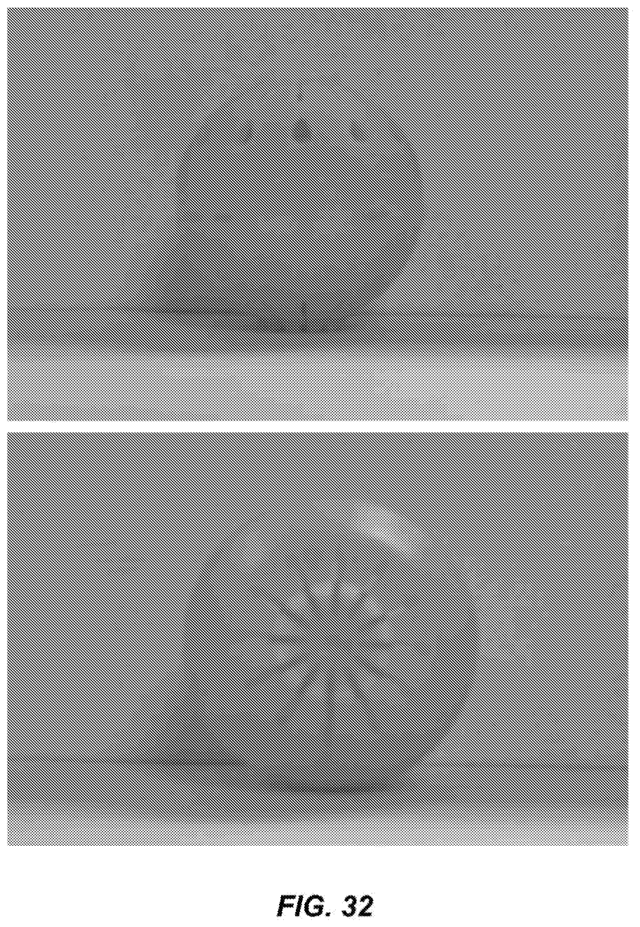

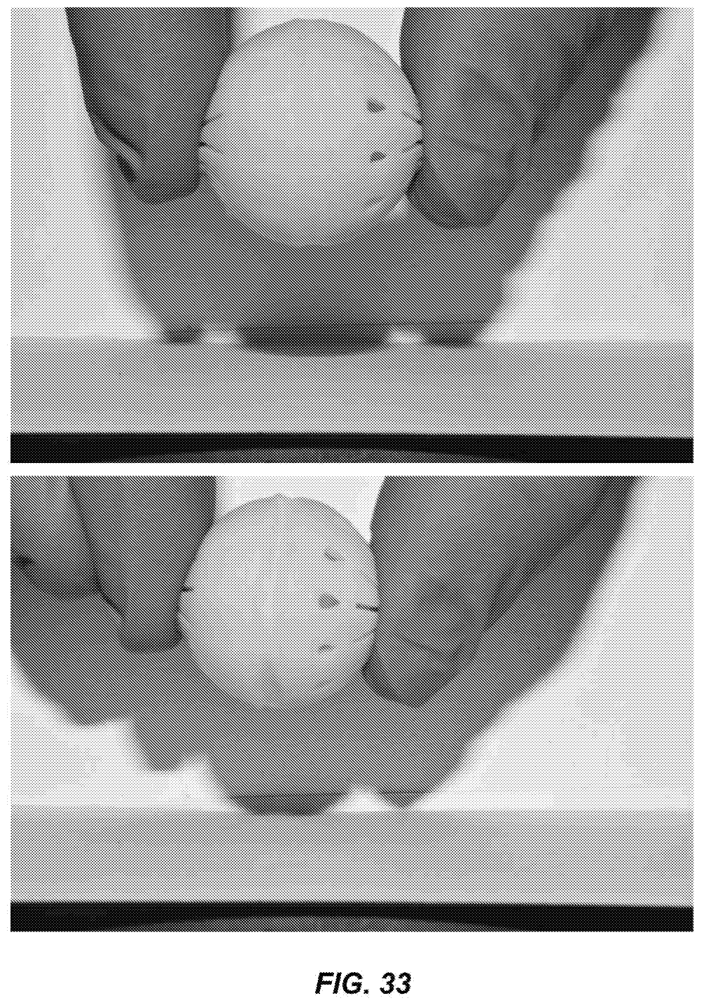

The structural unit can be a variety of shapes and sizes depending on the functional properties desired. The structural unit can be a symmetric shape or can be an asymmetric shape. For example, a conical shaped unit can be used to create convergent channels for a fluid, as described herein. In embodiments, the fluid comprises a bioactive agent. As another example, an oval shaped unit (when compared to a spherical unit) can be used to restrict the volume available to a fluid while maintaining same conduit section in correspondence of the transverse section. Non-limiting examples of shapes that embodiments can comprise include a sphere, a conical shape, an oval shape, a cubical shape, or an egg shape. The sphere, for example, can comprise a diameter of approximately 50 μm to approximately 2000 μm. As another example, the shape can comprise a volume of approximately 100 μm³ to approximately 10,000 μm³, such as approximately 6500 μm³. See, for example, FIG. 30, wherein the structural unit comprises diameters of 50 μm and 25 mm.

As desired, the structural unit can be an asymmetric shape or can be a symmetric shape. Asymmetric shapes can be used to have non orthotropic behaviors, for example, non-homogenous deformation of the unit or non-uniform volume for the fluid surrounding the unit. In embodiments, the structure displays non-axial symmetry, such as a structure that is not axially symmetric.

Referring to FIG. 2, for example, the structural unit can be sub-divided into different hemispheres. In embodiments, the hemispheres are structurally identical, structurally similar, or structurally different. The skilled artisan will recognize that whether the hemispheres are structurally identical or structurally different will depend on the desired function of the structural unit.

The structural unit can be composed of one or more biocompatible materials e.g., such that it results in no induction of inflammation or irritation when implanted. As used herein, “biocompatible” refers to a material which is not toxic, not injurious or not inhibitory to mammalian cells, tissues, or organs with which it comes in contact. Furthermore, when the material is in use with respect to the bioscaffold does not induce an immunological or inflammatory response sufficient to be deleterious to the subject's health or to implantation of the scaffold. Non-limiting examples of materials that embodiments can comprise include a photoresist polymer or a polymer that is compatible with three-dimensional printing technology, such as (2-(Hydroxymethyl)-2-[[(1-oxoallyl)oxy]methyl]-1,3-propanediyldiacrylate, known as IP-L; Photoresist pentaerythritol tetraacrylate (PETTA, Sigma-Aldrich) containing 3% Irgacure 379 photoinitiator (Ciba); polyurethanes polycaprolactone, polyglycolic acid, polylactic acid, polyamides, polyolefin, polyester, polytetrafluoroethylene, polyurethanes, and hydrogels used for bioprinting such as collagen, alginate, agarose, and chitosan, and synthetic hydrogels such as hyaluronan-methylcellulose, polyethylene glycol diacrylate collagen, laminin, matrigel, and non-biodegradable materials such as polysiloxanes, Stainless steel, Co—Cr alloys, Ti-alloys.

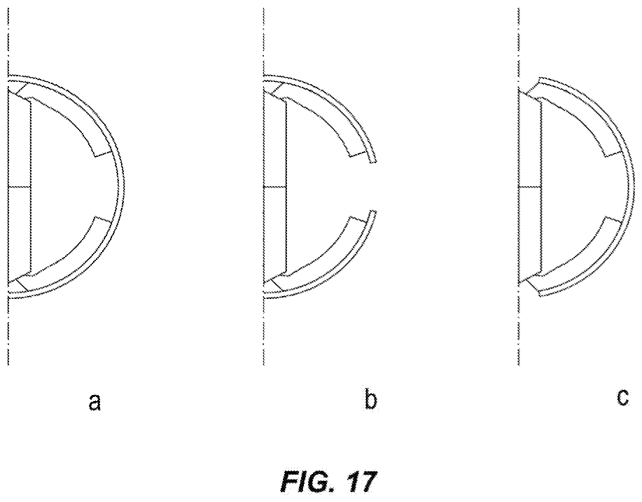

Referring to FIG. 2 and FIG. 3, for example, the structural unit can optionally comprise an external extended surface (also referred to as an “external shell” or simply “shell” (s)) surrounding the structural unit. The external extended surface, or shell or external shell, can provide an inner region and an outer region. The external shell can be fully sealed or contain one or more openings (op), such as in the superior and inferior portion of the units. When fully sealed, fluids, such media such as DMEM Media, DMEM/F12 Media, Ham's F-10 and F-12 Media, Medium 199, MEM, RPMI 1640 Media, Serum, can be included/encased inside (i.e., within the inner region) to have mechanical behaviors regulated by the laws of thermodynamics. Alternatively, the openings can, for example, be used to regulate the biodegradability of the components of the inner region of the unit, establish equilibrium between external and internal pressures, can be used to gradually supply nutrients or substances of any kind, can be used to release medications over time when implanted in vivo, supplies substances including nutrients, fit or connect two or more structural units, or can be simply used to filter a stream of particles surrounding the unit. In a bioscaffold, for example, the openings between two or more structural units can be in communication with each other, such as fluid communication. FIG. 17, for example, provides exemplary embodiments of a structural unit partial coverage by the external shell, such as when there is an opening in correspondence to the transverse middle plane or in correspondence to the two openings. As desired, the openings can be flat and/or radially extended. It will be understood that the openings in the shells can be present in various quantities, shapes and dimensions, which can be used to determine biodegradability of the unit, substance release, regulate stress concentrations, and fine tune permeability, for example.

The thickness of the external shell can be varied dependent on a desired functional property, such as biodegradability, hardness, density, compressibility, and the like. Referring to FIG. 19, for example, the external shell can be characterized by a constant thickness surrounding the structural unit, a thickness that varies around the structural unit (such as in relation to the polar coordinates), or localized variation of thickness (thinning or thickening), such as to exhibit localized deformations or to guarantee durability of the structure in the points mainly subject to stress.

The external shell can be formed of one or more materials. Non-limiting examples of such materials comprise Polylactic acid (PLA), Poly glycolic acid (PGA), Poly (lactic-co-glicolic acid) (PLGA), Poly ε-caprolactone (PCL), Polyethylene glycol (PEG), Polybutylene terephthalate (PBT), Polyethylene terephthalate (PET), Polyvinyl alcohol (PVA), Poly propylene fumarate (PPF), Poly aldehyde guluronate (PAG), polyacrylic acid (PAA), Polyurethane (PUR & PU), Collagen (type I, type II, type III), Alginate, Chitosan, Chitin, metals, and the like.

The external shell can also be composed on one or more layers. Referring to FIG. 21, for example, the one or more layers can be concentric, partially distributed, with holes, or limited to certain portions. Such variations can be used, for example, to modulate biodegradability, stiffen certain regions, or simplify manufacturing.

In embodiments, the external shell can be articulated. For example, such articulation of the external shell can be instrumental to increase surface of contact with cells, reduce stress concentration during deformation, drive local path of the deformation, and/or create cases of perfect fitting with surrounding units for specific states of deformation.

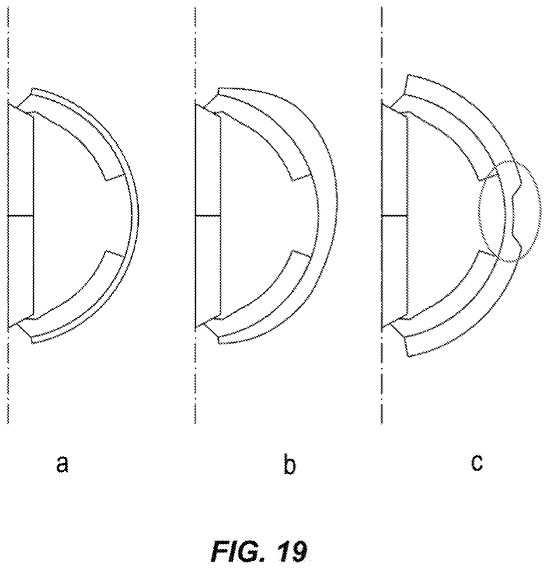

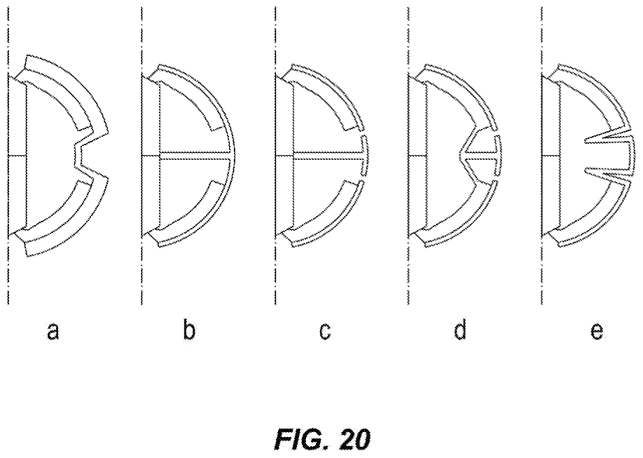

Referring to FIG. 20, for example, the external shell can have a profile recessed in the unit, can be directly connected to the central pillar with or without external continuity, or can have complex connections to the ribs, such as to accommodate compression without stress concentrations or to fine modulate its deformation.

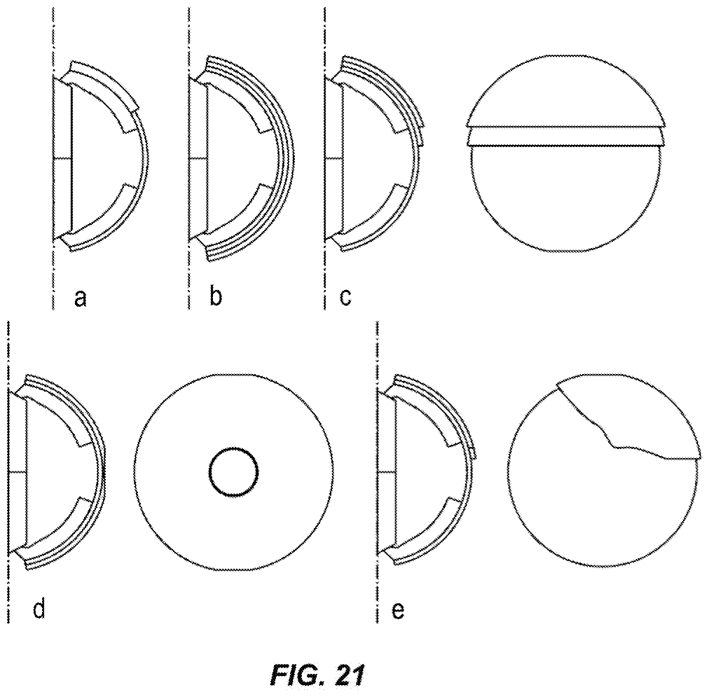

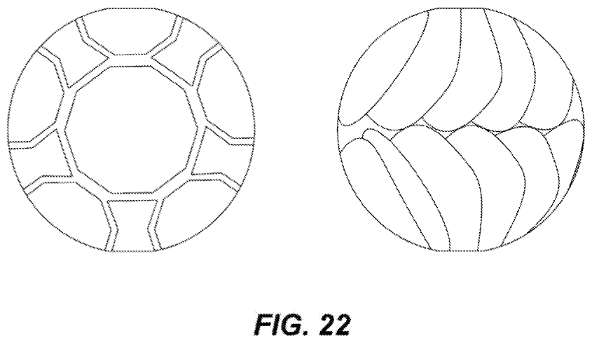

In embodiments, the external shell can be composed of a single part (i.e., a single external shell), or can be composed of two or more independent parts. See, FIG. 22, for example. Such configurations, for example, would ease deformation of the unit while adopting a rigid material. Such configuration, for example, can also allow deformations of the shell to be isolated to single parts, allow the spacing created between parts for certain loads can be used and calibrated to expose the inside the unit to the outside (drug release, biodegradability, etc etc), and/or be manufactured from the assembly of multiple components.

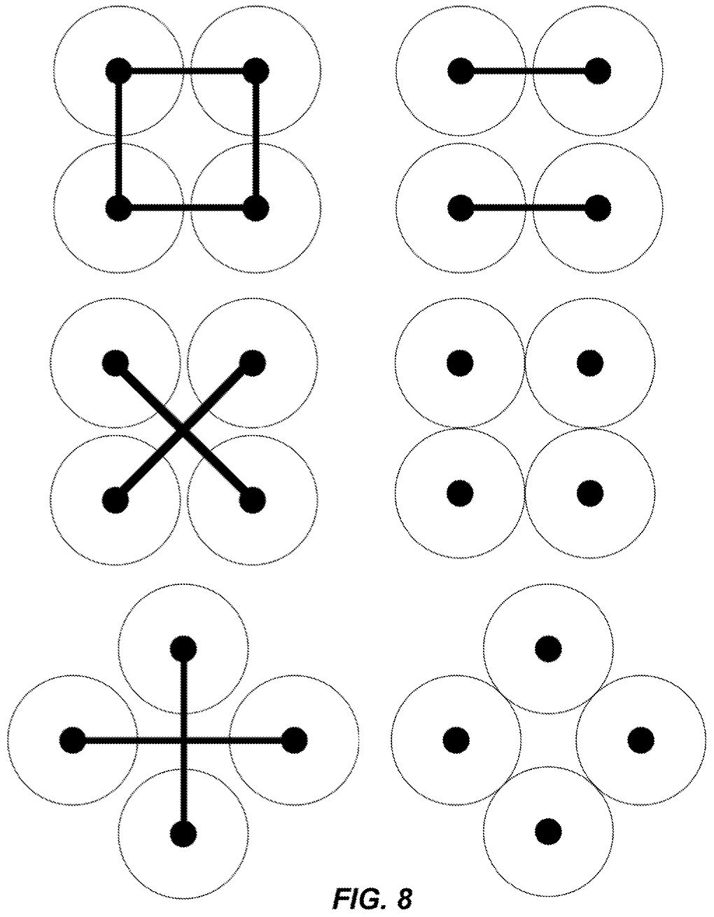

In embodiments, the structural unit can comprise connecting elements (“ce”) that connect the plurality of load arms to the central pillar. Connecting elements can be used, for example, to preserve the distances between the structural units independently from the applied axial load. In other embodiments, structural units can be directly attached one to another to have a scaffold transverse deformation controlled by the units expansion or contraction (see FIG. 8). Connecting elements can be manufactured from a material identical to or stiffer than that of the core structural unit. Such materials comprise metallic materials.

One or more structural units can suspended or randomly dispersed in a media, such as a fluid, spray, solid, semi-solid, gel, or powder, to provide a composition. In embodiments, the media is a pharmaceutically acceptable media, such as a pharmaceutically acceptable carrier. In embodiments, the media comprises matrigels and/or hydrogels. As such, the composition is suitable to be administered to a subject. In embodiments, the structural units can be embedded in an object, such as a bone screw, prosthesis or corresponding coating.

For example, a composition comprising the structural units, such as a coating, can be applied to a device in any suitable fashion, e.g., it can be applied directly to the surface of the medical device, or alternatively, to the surface of a surface-modified medical device, by dipping, spraying, or any conventional technique. The method of applying the coating composition to the device is typically governed by the geometry of the device and other process considerations.

The structural unit can be manufactured using techniques known in the art, such as utilizing three-dimensional printing or assembling pre-built components, such as pre-built arms and ribs. The structural unit can be manufactured by coupling two partially constructed units, such as two semi-hemispheric units.

Bioscaffold

Aspects of the invention are directed towards a scaffold comprising at least two structural units that are connected to, such as by connecting elements, and/or in communication with each other. Referring to FIG. 8, the structural units can be connected in a variety of orientations, each of which can confer structural/functional properties to the bioscaffold, including hydrophobicity, durability, bioactive agent release characteristics, biocompatibility, molecular weight, availability, and cost.

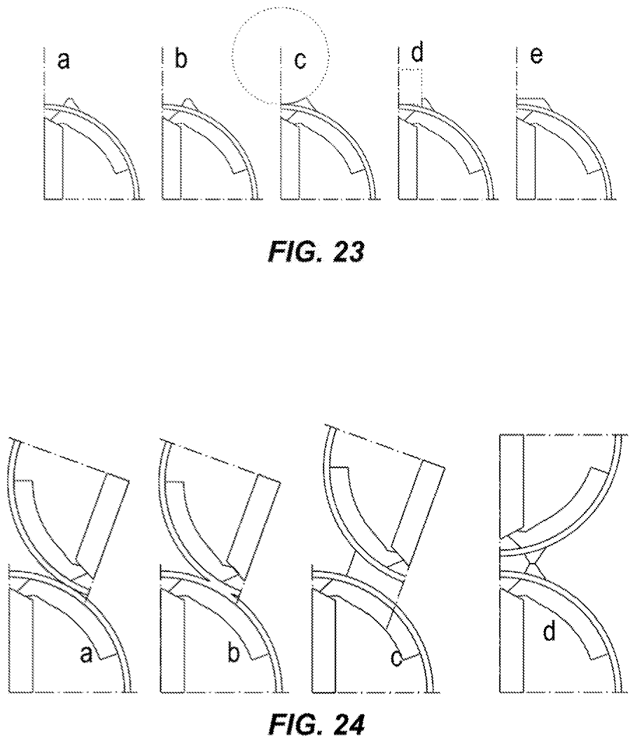

For example, the structural units can comprise one or more supports which facilitate unit organization. Referring to FIG. 23, for example, the superior and inferior portions of the unit can be comprise one or more supports that can be used to stabilize the unit within the scaffold, to increases the torque on the ribs or simply to link multiple ribs that can be actuated simultaneously. For this functionality, these supports can also be of different heights within a single unit. As illustrated in FIG. 23, for example, the supports can be made with a flat surface, rounded edges, describe a circular or elliptical surface, able to enclose a cylinder, or simply flat in relation to the desired connection. By altering the configurations of the supports, the loading of the unit can also be influenced.

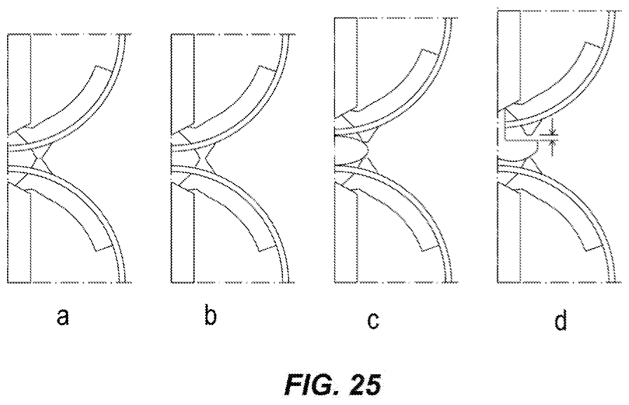

Referring to FIG. 25, for example, the supports can be of simple contact between the units or can be bonded to constitute a continuum. When there is a third element interposed, the shape of this element can determine the modality of deformation of the unit, following an elliptical path for both units, following an elliptical path on one unit while the other is actuated only after the load needed to close the gap is achieved. Note that the interpose element and also the units are not necessarily axisymmetric despite the drawings.

Two or more structural units can be organized as in various manners and patterns to provide a desired functionality. Referring to FIG. 24, for example, the multiple units can be in simple contact, bonded, have a third element interpositioned in between two individual units, or through supports or connecting elements.

In simple contact, the units can rigidly move and their loading can be stochastic. By modulating surface material properties and roughness, the shear force transmitted can be manipulated.

When bonded, the units are constrained one to each other so the relative movements are driven purely by deformations.

When elements are interposed, one can design the surface of contact according to the geometry of the element, for example to reduce pressure and avoid stress concentrations.

Referring to FIG. 25, for example, when elements are interposed between supports the profile of the intermediate element drives the contact and of consequence the deformation of the rib.

The disposition of the structural units can be dictated by the desired functionality. For example, referring to the disposition of the structural units as a bone scaffold, the disposition of the structural units can be dictated by the desired bone profile to be achieved, and the dimensions and components of the structural units can be varied within a structure in relation to the desired bone density to be achieved. Both profile and density, in addition to be arbitrarily chosen functions, can be taken from existing CT images, can be obtained as result of stress or fluid dynamic analysis, or simply dictated by limitations of the manufacturing process, for example.

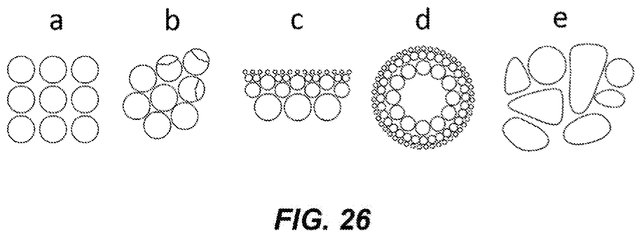

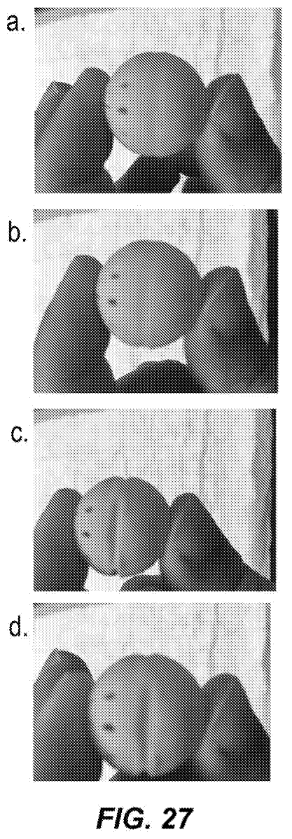

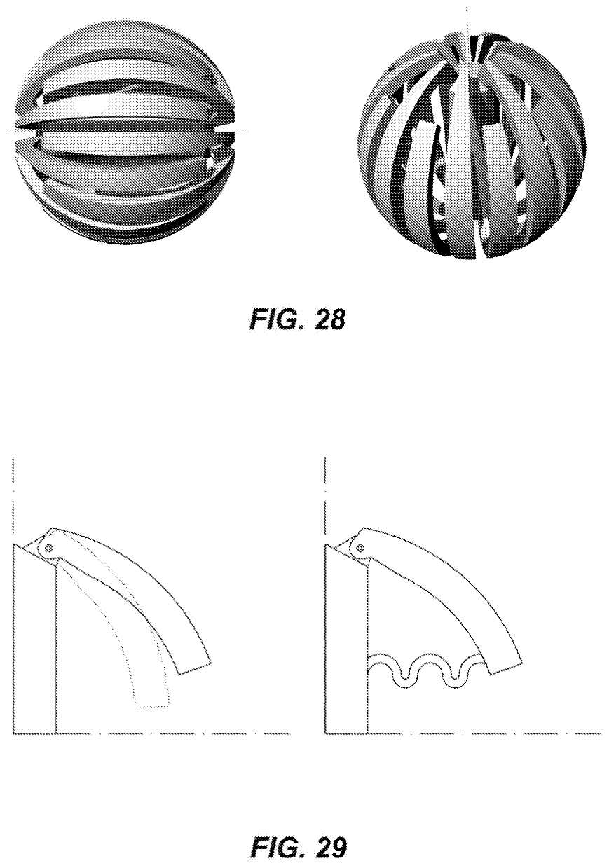

Referring to FIG. 26, for example, examples of scaffolds dispositions can be drawn with patterns organized as a matrix, honeycomb, in layers of different unit dimensions disposed in plane, concentric, simply composed by various shaped units not organized following an organized disposition or progression in their dimension, and the like.

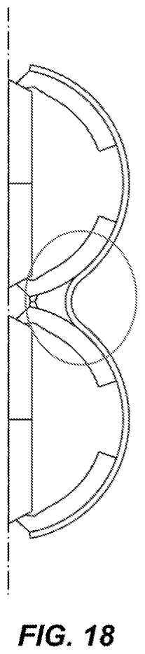

Referring to FIG. 18, for example, the scaffold can comprise an external shell that is continuous on one or more structural units. For example, the external shell can be fabricated with an elongated profile that works as a sleeve for one or more units.

In embodiments, the scaffold comprises a bioscaffold or a tissue scaffold for in vivo use, such as for implantation into a subject. In presence of large tissue defects, such as large defects of bone, tendons, ligaments, cartilage, and the like, the bioscaffold can serve as a matrix for the regeneration of tissue. For example, the bioscaffold can store and/or release bioactive agents, nutrients, water, cell survivability enhancers and/or growth factors; provide mechanical stimuli; and/or induce cell proliferation.

In a preferred embodiment, the bioscaffold is constructed so as to avoid immunological responses (i.e., biocompatible) such that it results in no induction of inflammation or irritation when implanted into a subject.

In other embodiments, the bioscaffold can be constructed of a biodegradable materials so as to degrade over a period of time, for example as cells proliferate and/or tissue regenerates. In other embodiments, the bioscaffold can be constructed of non-biodegradable materials.

As described herein, the bioscaffold can host cells and nutrients so as to assist and/or promote tissue regeneration. For example, the bioscaffold can serve as a foundation upon which cells can adhere to and proliferate, thus assisting in the regeneration of tissue.

The bioscaffold can have the structural, mechanical and functional properties described herein while at the same time be able to withstand physiological loads. For example, embodiments can comprise new three dimensional scaffolds for extended tissue reconstruction that are able to withstand physiological loads. In embodiments, the scaffolds demonstrate enhanced cell survivability.

When compared to scaffolds of the largely adopted truss concept, the bioscaffold described herein can have higher adhesion surface and lower porosity to more precisely modulate nutrients flow. In embodiments, the bioscaffold and/or structural unit can be porous, so as to absorb and/or release fluids. For example, the scaffold can comprise at least one bioactive agent, which can be absorbed or released from the bioscaffold.

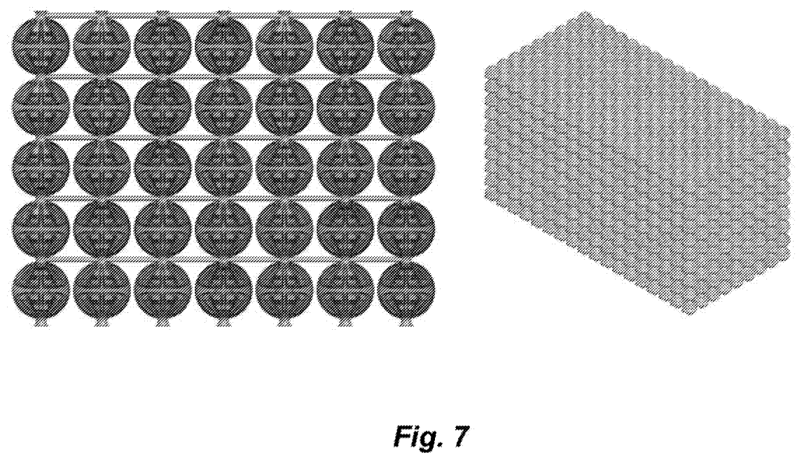

The scaffold can be provided as a variety of shapes and sizes so as to provide an optimal combination of various structural/function/mechanical properties, including durability, bioactive agent release characteristics, cell survivability, availability, cost, and the like. Non-limiting examples of such shapes comprise a sheet, a stack, a spiral, or a linear bioscaffold. For example, the scaffold can be provided as a flat organization in a spiral so as to easily be adapted to cover bone screws. As another example, the scaffold can form helicoidal shapes. Referring to FIG. 7, for example, the bioscaffold can be provided as a two-dimensional sheet. The two-dimensional sheet can be used, for example, as a coating for an object, such as a bone screw or prosthesis. Alternatively, the two-dimensional sheets can be used in the manufacture of a three-dimensional scaffold, such as a stack. a sheet of structural units can be layered to form a three-dimensional stack. For example, the bioscaffold can be manufactures from one or more sheets, such two sheets, three sheets, four sheets, five sheets, six sheets, seven sheets, eight sheets, nine sheets, ten sheets, or more than ten sheets. See FIG. 7, for example, wherein the bioscaffold comprises 10 sheets. The three-dimensional bioscaffold can be provided, for example, by folding or rolling two-dimensional layers of units to provide a three-dimensional scaffold. Alternatively, the two-dimensional layers can be stacked to provide a three-dimensional scaffold.

As described herein, Connecting Elements can be used to preserve the distances between structural units independently from the applied axial load, otherwise units can be directly attached one to another to have a scaffold transverse deformation controlled by the units expansion or contraction (see FIG. 8).

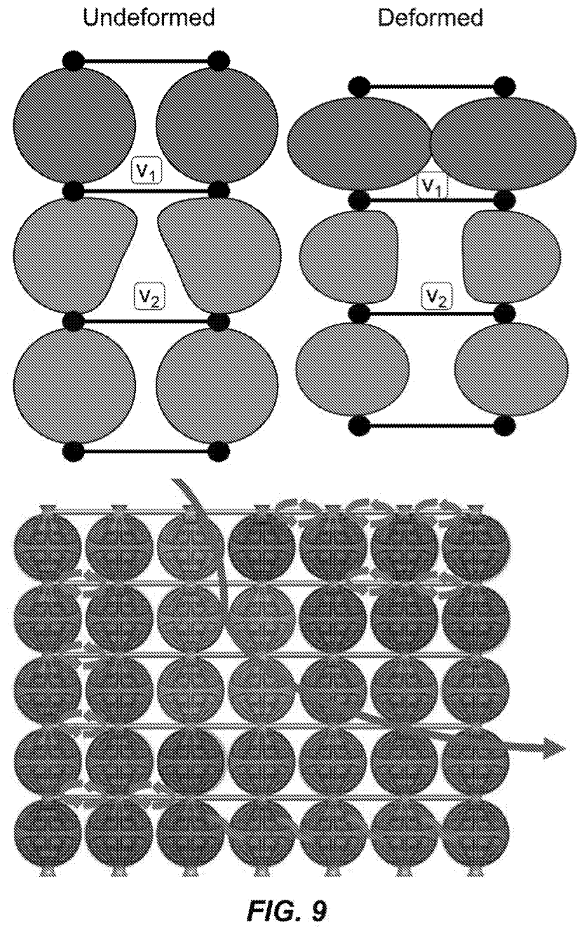

The scaffold can comprise structural units that are identical in their mechanical, structural, and/or physical properties, similar in their mechanical, structural, and/or physical properties, or different in their mechanical, structural, and/or physical properties. These differences can convey optimal functional properties to the scaffold, such as permitting the bioactive or fluid release rate to be adjusted and controlled and/or enhancing cell survivability. For example, the bioscaffold can comprise structural units with different auxetic behaviors organized in a fashion so as to generate pressure gradients in the scaffold able to drive the nutrients flow within the scaffold (see FIG. 9).

Existing scaffolds, for example, have low inner survivability and their application is limited to small bone defects. “Vascularized scaffolds”, as embodiments described herein can be referred to as, can generate gradients to move nutrients toward the inner deep portions of the scaffold so there is uniform bone growth, thus allowing embodiments to be used to for large tissue defects, such as extended bone defects. In other embodiments, the scaffolds can be designed so as to release bioactive agents or fluids during physical activity when implanted in vivo. Alternatively, the scaffolds can be designed for the time-dependent release of bioactive agents or fluids over a period of time.

The scaffold can further comprise elements that are able to expand or contract independently from the applied loads, such as body loads, so as to allow for the regulation of nutrient and/or fluid flow within the scaffold. Non-limiting examples of such elements comprise memory alloy, electric motors, units that react to external magnetic fields, those that reacts to changes in the environment (such as body temperature), units with combination of mass and stiffness that exhibits large displacement under variable loads.

The term “load” can refer to the force exerted on an object, such as the force exerted on the structural unit or scaffold comprising the same. A non-limiting example of such applied load comprises a body load. For example, muscle contraction is a type of body load that will put pressure on the structural unit/scaffold in one or more directions. For example, the body load can be along the axis of a structural unit, not along the axis of the structural unit, or a combination thereof. Structural units can be designed to withstand more than one applied loads, such as more than one body loads.

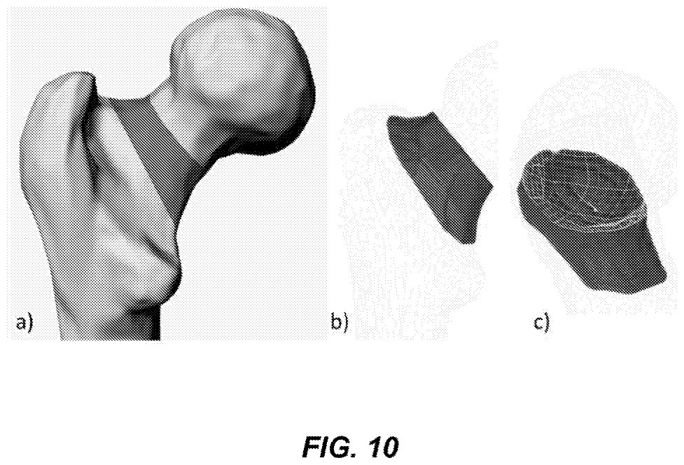

To address reconstruction of largely extended bone defects (see FIG. 10), an algorithm was developed that, with bone geometry and blood vessels reconstructed from MRI, can generate appropriate scaffold geometry and localized auxetic behaviors to maximize cell survival.

In embodiments, the bioscaffold can be seeded with viable cells so as to populate the bioscaffold with the viable cells. The term “viable cell” can refer to a cell that is alive and capable of growth, proliferation, migration, and/or differentiation. The bioscaffold can act as structural scaffold upon which viable cells can migrate and readily repopulate. In some embodiments, cells from the native tissue (e.g., the host subject) can also migrate into the bioscaffold and readily repopulate the polymer-permeated graft in vivo.

For example, the bioscaffold can be seeded and incubated with exogenous cells under conditions conducive to populating the bioscaffold with the exogenous cells or cells derived from the exogenous cells. In some embodiments, the exogenous cells can be autologous, homologous (e.g., allogenic), or heterologous. For example, “autologous” refers to biological material (e.g., exogenous cells) that will be introduced into the same individual from whom the material was collected or derived. For example, “homologous” can refer to biological material (e.g., exogenous cells) collected or derived from a compatible donor that will be introduced into a different individual from which the material was collected or derived. For example, “heterologous” can refer to biological material (e.g., exogenous cells) collected or derived from a compatible donor of a different species that will be introduced into an individual. Non-limiting examples of cells that can be seeded onto (and thus useful for populating the bioscaffold) include osteoblasts, osteoclasts, lining cells, keratinocytes, melanocytes, nerve cells, stromal cells, fibroblasts, endothelial cells, progenitor cells, stem cells, organ-specific cells, tissue-specific cells, or a combination thereof.

Conditions conducive to populate the scaffold are dependent upon the cells used, and can include temperature, the presence or absence of growth factors, the presence or absence of differentiation factors or migration factors, or the air content. In embodiments, the bioscaffold is introduced or implanted into a subject, and the subject's own cells migrate into the graft. In other embodiments, viable cells are introduced into the bioscaffold prior to implanting the graft onto the subject.

One of skill in the art can seed exogenous cells onto the bioscaffold by placing the bioscaffold into culture medium containing dissociated, or dissociated and expanded, cells and allowing the cells to migrate into the bioscaffold and populate the bioscaffold. In some embodiments, cells can be injected into one or more places in the bioscaffold, such as into the interior, in order to accelerate repopulation of the structures.

The viable cells can be cultured prior to populating or seeding of the bioscaffold. Culture mediums used to grow and expand cells of interest is cell-type-dependent, and is known to those skilled in the art. The culture medium can be serum-free and would not require the use of feeder cells.

The scaffold can comprise at least one bioactive agent, which can refer to virtually any substance which possesses desirable characteristics for application to the implant site. For example, the scaffold can be coated with the at least one bioactive agent or can contain the bioactive agent so as to release the bioactive agent within a subject. The bioactive agents useful in the present invention include thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti-secretory agents, actin inhibitors, remodeling inhibitors, antisense nucleotides, anti-metabolites, antiproliferatives, anticancer chemotherapeutic agents, anti-inflammatory steroid or non-steroidal anti-inflammatory agents, immunosuppressive agents, growth hormone antagonists, growth factors, vitamins, cell viability enhancers, dopamine agonists, radiotherapeutic agents, peptides, proteins, enzymes, extracellular matrix components, ACE inhibitors, free radical scavengers, chelators, antioxidants, anti-polymerases, antiviral agents, photodynamic therapy agents, and gene therapy agents.

“Cell Viability Enhancer” can refer to a substance that enhances and/or promotes the viability and/or growth of a cell.

“Antibiotic” can refer to a substance that controls the growth of bacteria, fungi, or similar microorganisms, wherein the substance can be a natural substance produced by bacteria or fungi, or a chemically/biochemically synthesized substance (which may be an analog of a natural substance), or a chemically modified form of a natural substance. One of skill will recognize that the scaffold can be coated with a wide variety of antibiotics, such as penicillins, cephalosporins, macrolides, fluoroquinolones, sulfonamides, tetracyclines, aminoglycosides, and the like.

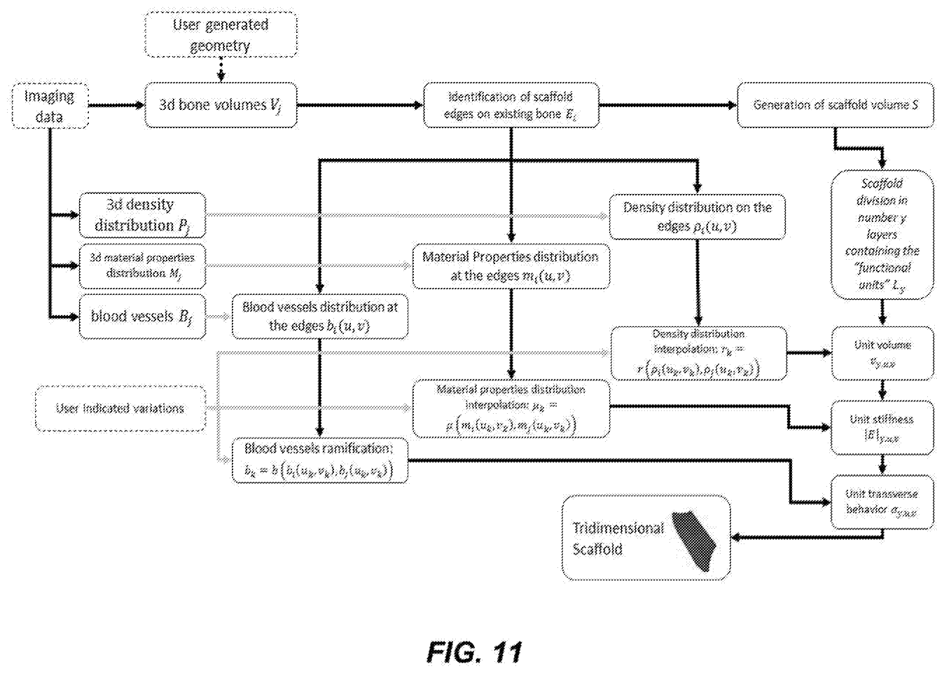

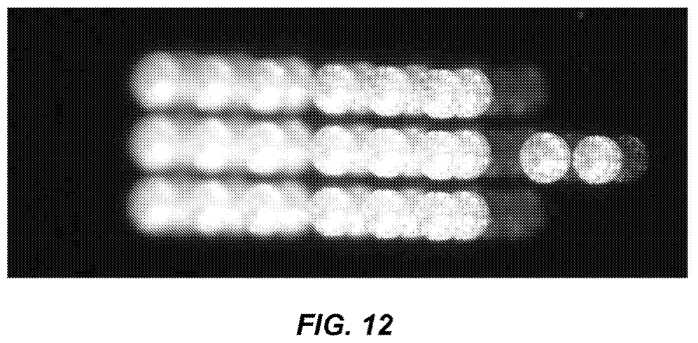

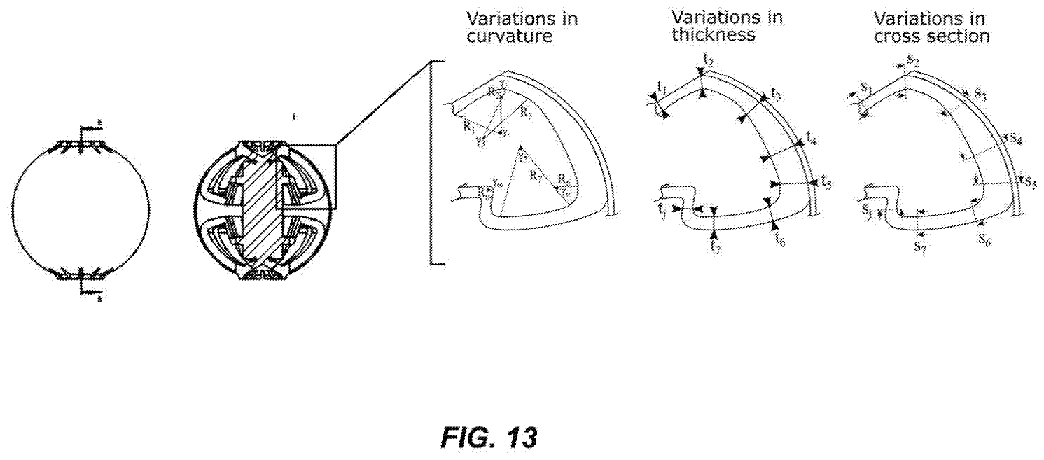

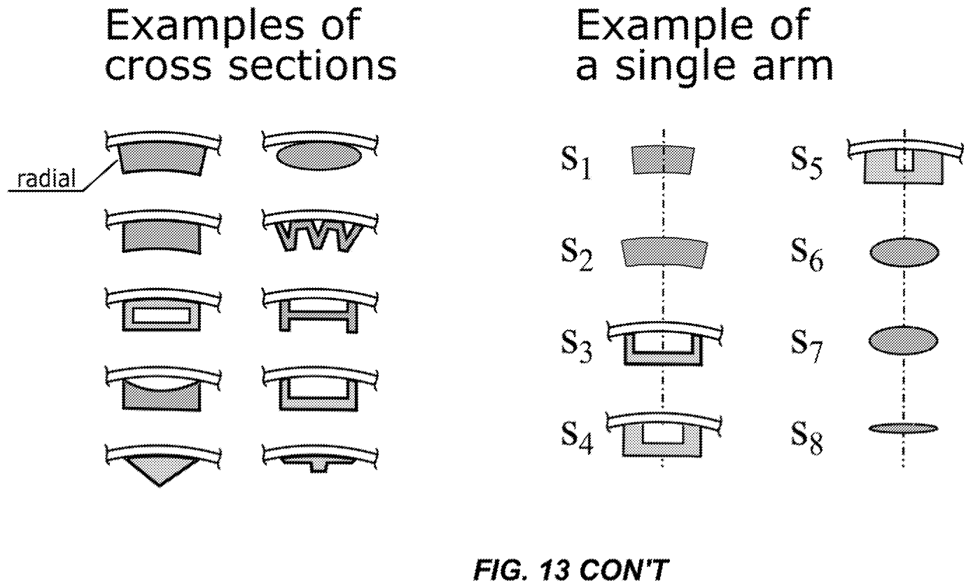

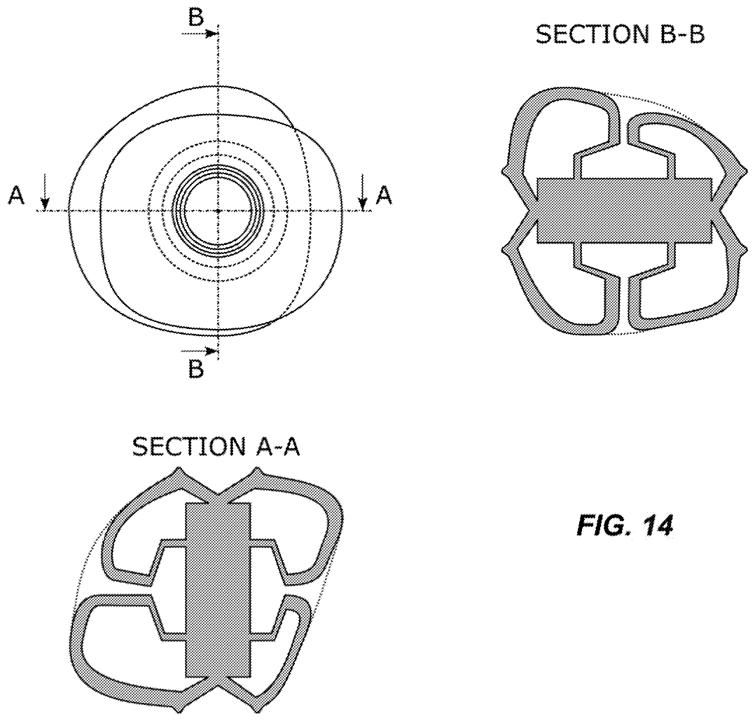

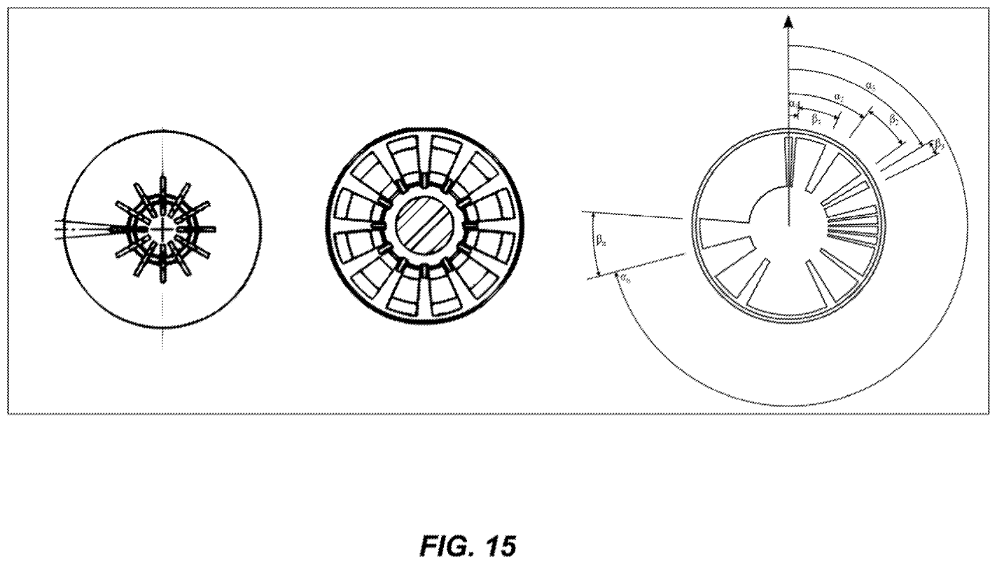

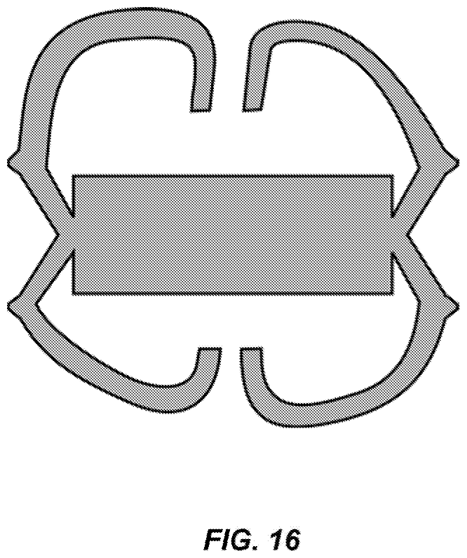

Aspects of the invention are also directed towards a method comprising a series of actions to evaluate the characterization of the structural and/or functional units in relation to their spatial position within the scaffold. Aspects of the invention are also directed towards a method for designing a bioscaffold. In embodiments, the methods can be provided as a computer-aided-design. See, for example, U.S. Pat. No. 7,747,305, which is incorporated by reference herein in its entirety. Such computer aided design methods can relate to the generation of patient-specific and/or tissue-specific structural units and/or scaffolds for in vivo use. In embodiments, the method can be provided as an algorithm. See FIG. 11, for example. The method can receive input manually (such as 3D geometries designed in a CAD or coordinates given in input to define geometric primitives) and/or from one or more elements obtained from diagnostic imaging, such as reconstructions of bone, blood vessels, material density, and structural properties distributions. Embodiments can further comprise the input of elements as copy of existing reconstruction that can be used on a specific patient with scaling or/and morphing, for example, as is the case of scaffold applied to subject A but obtained from CT data of subject B.

Method of Regenerating Tissue

Aspects of the invention are directed towards methods of using a bioscaffold to treat a subject in need thereof or regenerating a tissue, such as bone, in a subject in need thereof. For example, the subject may be in need of repair and/or replacement of bone or a tissue (such as a ligament, tendon, cartilage, muscle, and the like). In embodiments, the method can comprise obtaining a bioscaffold described herein and securing/implanting the bioscaffold to a prepared site on or within the subject. In some embodiments, the method further comprises allowing time for cells from the subject to integrate into the scaffold. In some embodiments, the method comprises populating the scaffold with cells prior to implantation into the subject. In some embodiments, the method further comprises allowing time for the scaffold to degrade.

The term “treating” can refer to partially or completely alleviating, ameliorating, improving, relieving, delaying onset of, inhibiting progression of, reducing severity of, and/or reducing incidence of one or more symptoms, features, or clinical manifestations of a particular disease, disorder, and/or condition. Treatment can be administered to a subject who does not exhibit signs of a disease, disorder, and/or condition (e.g., prior to an identifiable disease, disorder, and/or condition), and/or to a subject who exhibits only early signs of a disease, disorder, and/or condition for the purpose of decreasing the risk of developing pathology associated with the disease, disorder, and/or condition.

As described herein, for example, the structural unit can release a bioactive/therapeutic agent so as to treat a subject in need thereof. In other embodiments, the structural unit and/or bioscaffold can serve as a foundation for a population of cells so as to regenerate or regrow bone or a tissue in a subject in need thereof.

The term “subject” or “patient” can refer to any organism to which aspects of the invention can be administered, e.g., for experimental, diagnostic, prophylactic, and/or therapeutic purposes. Typical subjects to which compositions of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted above or another organism that is alive. The term “living subject” refers to the entire subject or organism and not just a part excised (e.g., a liver or other organ) from the living subject.

As used herein, “changed as compared to a control” sample or subject is understood as having a level of an analyte or diagnostic or therapeutic indicator (e.g., marker) to be detected at a level that is statistically different than a sample from a normal, untreated, or abnormal state control sample. The diagnostic or therapeutic indicator can be assessment of the growth of the tissue grafted or observation for lack of graft rejection. Determination of statistical significance is within the ability of those skilled in the art, e.g., the number of standard deviations from the mean that constitute a positive or negative result.

In embodiments, the bioscaffold can be implanted onto a prepared site on or within a subject in need thereof; thereby implanting to a subject the bioscaffold.

In embodiments, the bioscaffold has been repopulated with viable cells as described herein.

Bioscaffolds of the invention can be maintained in a cell culture medium suitable for maintenance and expansion of cells. The culture medium used to grow and expand cells of interest can be serum-free and would not require the use of feeder cells.

Kits

The compositions and scaffolds as described herein can also be provided in a kit. In one embodiment, the kit includes (a) a container that contains a composition, one or more structural units, and/or a scaffold as described herein, and optionally (b) informational material. The informational material can be descriptive, instructional, marketing or other material that relates to the methods described herein and/or the use of the composition, the scaffold for therapeutic benefit, or solutions.

The informational material of the kits is not limited in its form. In one embodiment, the informational material can include information about production of the composition, structural unit, or scaffold, components of the same, date of expiration, batch or production site information, and so forth. In one embodiment, the informational material relates to methods of administering or affixing the composition or scaffold, e.g., in a suitable form, or mode of administration, to treat a subject. The information can be provided in a variety of formats, include printed text, computer readable material, video recording, or audio recording, or information that provides a link or address to substantive material. The composition or scaffold can be provided in a sterile form and prepackaged.

The kit can include one or more containers for the composition or scaffold described herein. In some embodiments, the kit contains separate containers, dividers or compartments for the composition or scaffold and informational material. For example, the informational material can be contained in a plastic sleeve or packet. In other embodiments, the separate elements of the kit are contained within a single, undivided container. For example, the scaffold is contained in a container that has attached thereto the informational material in the form of a label. The containers of the kits can be air tight, waterproof (e.g., impermeable to changes in moisture or evaporation), and/or light-tight.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Tridimensional Bone Scaffolds for Extended Bone Reconstruction Able to Withstand Physiological Loads with Enhanced Cell Survivability

In presence of large bone defects, a scaffold serves as a matrix for the regeneration of tissue. Scaffold stores nutrients, water, growth factors, and induces cell proliferation. To address this purpose a scaffold should avoid immunological responses, degrade as cells proliferate, and host cells and nutrients. All these requirements should be fulfilled within a structure that is able to withstand physiological loads.

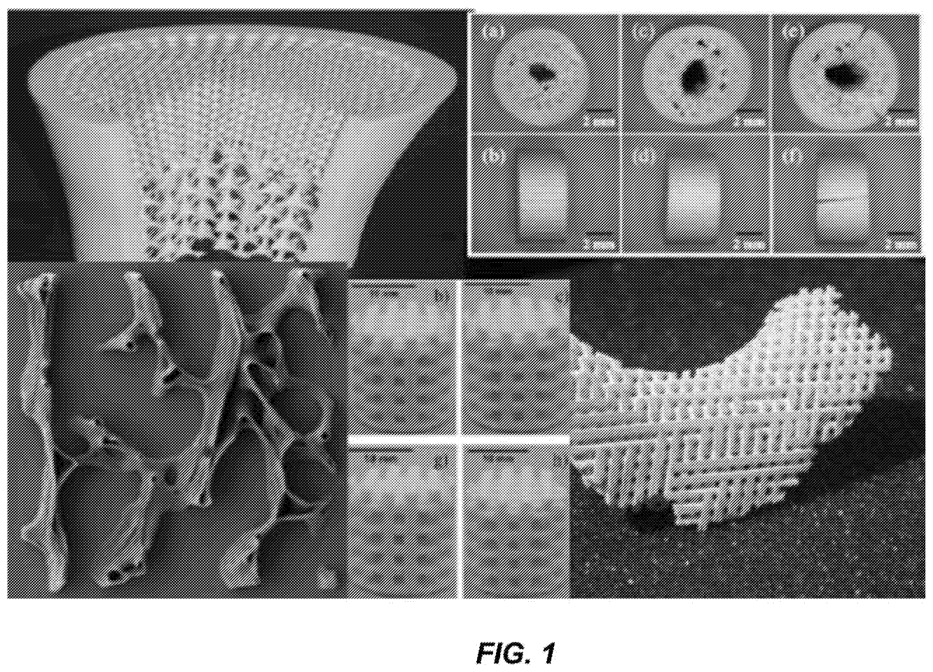

In current approaches, biocompatibility, degradability, and cell adhesion are given to the scaffolds through the materials composing the scaffold using synthesis of a bioceramic materials such as hydroxyapatite or synthetic polymers such as PLA. Cell hosting and nutrition are conferred through porosity and scaffold designs. Existing scaffolds are mostly created by imitating bone architecture (FIG. 1), at the point that the scaffold is obtained through segmentation of microCT data. The trabeculae are modelled as beams and cell and nutrient hosting is required to the obtained trusses which are characterized by small adhesion surface and high porosity. Compared to actual bone, nutrient transport and mechanical stimuli are not locally regulated.

Embodiments described herein comprise new three dimensional bone scaffolds, such as those for extended bone reconstructions, that are able to withstand physiological loads with enhanced cell survivability. As described herein, the largely adopted truss concept has been overturned by proposing a scaffold that has higher adhesion surface and low porosity to precisely modulate nutrients flow. Considering the incompressible nature of the cells, under physiological loads, the scaffold should locally reduce its volume to avoid any undesired leakage. Auxetic structural units, such as that in (FIG. 2(a)), serves this purpose. In embodiments, the unit can be characterized by an external extended surface to guarantee cell adhesion. In common with bone, the unit can be designed to exhibit non-isotropic behavior and under axial load reduces its cross section and consequentially its volume. Considering that the Poisson's ratio expresses the proportion between axial and transverse deformations, a structure or material that exhibits this behavior would have a negative Poisson's ratio and in literature such material is indicated as auxetic.

Auxetic materials are well documented in existing literature and are used in several fields, including bone regeneration. However, the designed structure described herein can be produced with materials having positive Poisson's ratio while retaining auxetic behavior. This is an original concept and new to our knowledge. The unit shown as a sphere is suitable of different shapes such as conical, oval, cubical, etc and shows the auxetic behavior due to its inner structure (see FIG. 2(b)).

Embodiments described herein can be composed of ribs (r) that under compression form the presence of the “bending arms” (a) which folds towards the center dragging the external shell (s) (FIG. 3). Variability in rib thickness can be used to limit deformation to specific regions of the ribs (c). The deformation of the ribs can be controlled by their geometrical dimensions, for instance thickness (e), angular width (f), length of the “bending arm” (a), and position of the “pivoting arm” (p).

The ribs can be equally spaced (d) and of constant dimensions in order to confer a transverse isotropic behavior to the unit. The ribs can also be unequally spaced with eventually unequal dimensions to conferee variable transverse behaviors and stiffness with reference to specific directions (see FIG. 4).

Ribs extensions can be adjusted in order to reduce the gap (g) and control the non-linear behavior in response to axial deformation.

While its Poisson's ratio is determined by the geometrical configuration conferred to the radial branches, following an initial deformation, stiffness to axial load can be regulated through the dimensions of the central pillar (cp).

The central pillar can have several shapes. In FIG. 3, it is illustrated as cylindrical. Other plausible shapes include polygon sections, hollow to allow flow, host a shaft for sliding, or a connection element such as a cable that can be used to limit the applied loads to only compression or to apply a preload on the unit.

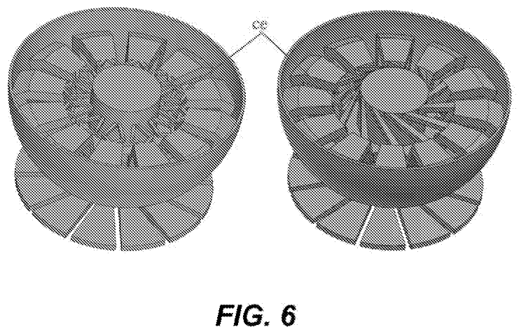

The superior and inferior portion of each unit are suitable to any shape. For example, FIG. 3 shows a structural unit with flat compact support to allow stackability. As another example, FIG. 6 shows a structural unit radially extended with circular shape to increase their stability to axial loads. This support (sup), supported by the ribs is divided radially to allow expansion or reduction in consequence respectively of compression or extension loads. The external shell can be fully sealed or contain openings (op) in the superior and inferior portion of the units. When fully sealed, fluids can be included/encased inside to have mechanical behaviors regulated by the laws of thermodynamics. Substances of different nature such as medications can be used for drug release when implanted in vivo. These openings can be used to establish equilibrium between external and internal pressures, can be used to gradually supply nutrients or substances of any kind or can be simply used to filter the stream surrounding the unit.

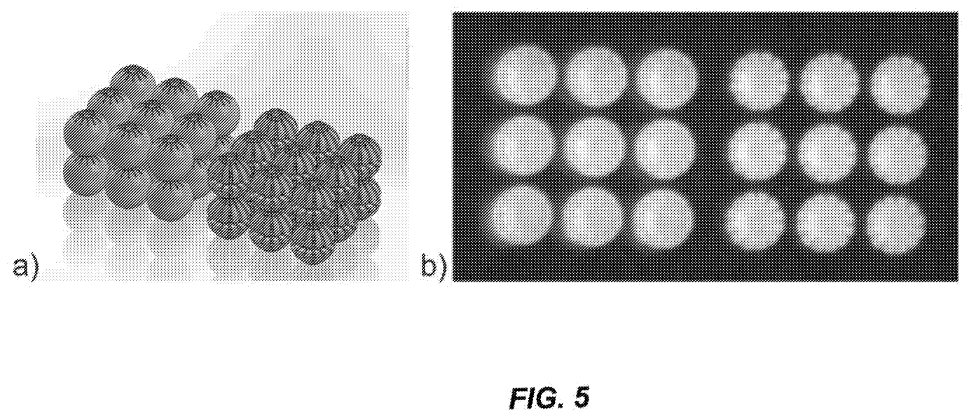

To prove that the structural units can be manufactured in three dimensions, a Nanoscribe 3D printer was utilized to create two staked units (100 μm in diameter) using a biocompatible photoresist polymer with and without external shell (FIG. 5).

In several 3D printing technologies, manufacturing is realized layer by layer and the inclusion of an additional connecting elements (ce) in proximity to the rib can be included to simplify the printing process (see FIG. 6).

The units can comprise sheets made of one or a few layers, or they can be partially or totally imbedded in layers created for different purposes, for example prosthesis coating. The units can be suspended in media of various nature such as fluids, solids, semi-solids, gels, or powders. The inclusion of the units in three dimensional structures, can be obtained by folding or rolling 2D layers of units or directly organizing the units in three dimensional matrices (see FIG. 7). Connecting elements can be used to preserve the distances between spheres/structural units independently from the applied axial load, otherwise units can be directly attached one to another to have a scaffold transverse deformation controlled by the units expansion or contraction (see FIG. 8).

The inclusion of units with different auxetic behaviors in organized paths could be used to generate pressure gradients in the scaffold able to drive the nutrients flow (see FIG. 9).

The inclusion of elements within these organized structures that are able to expand or contract independently from the applied loads can allow for the regulation of nutrient and/or fluid flow. Non-limiting examples of such elements can be units that contains memory alloy, electric motors, units that react to external magnetic fields, units with combination of mass and stiffness that exhibits large displacement under variable loads. To address reconstruction of largely extended bone defects (see FIG. 10), an algorithm was developed that, with bone geometry and blood vessels reconstructed from MRI, can generate appropriate scaffold geometry and localized auxetic behaviors to maximize cell survival.

The algorithm includes a series of actions performed with or without the user input to evaluate the characterization of the functional units in relation to their spatial position within the scaffold (See FIG. 11).

The algorithm can receive input manually, and/or from one or more elements obtained from diagnostic imaging, such as reconstructions of bone, blood vessels, material density, and structural properties distributions. These elements are shown in FIG. 11 with dashed lines to highlight that they can be provided by a user whenever they are not partially or totally obtained from imaging. The algorithm can be compatible with CAD software known in the art to, for example, supply a reconstruction of the scaffold geometry. On this geometry, after identification of the scaffold edges, interpolation and extrapolation are obtained the distribution maps on the edges of density, structural properties and blood vessels. Using user defined functions, the values at the edges can be interpolated to obtain the distribution of such properties within the scaffold. The interpolation can be performed following several strategies, for example, to interpolate points on the edges having similar values, or points having homologous position with functions that can represent gradients of different nature such as linear or quadratic. The tridimensional scaffold is obtained intersecting the created spatial distributions with planes sectioning the scaffold to assign for each point of the layer, the needed volumes, stiffness and transverse behavior of the corresponding unit.

### Example 2

Embodiments of the invention are directed towards an auxetic bone scaffold. The scaffold can be obtained with organizations of single units in complex structures. Therefore, embodiments comprise the single unit, the organization of the single units to form the scaffold, and how the organization can be framed in an algorithm for patient specific applications.

Single Unit

Compared to the trusses largely adopted as unit for bone scaffold, embodiments described herein have larger cell adhesion surface. Embodiments allow for deformation and preserve mechanical stimuli of the cells without reducing their vital space.

The auxetic behavior can be limited to a certain range of axial deformation beyond which the structure can exhibit a positive Poisson's ratio.

The axial stiffness and strength can be independent from transverse stiffness and strength.

Given the axial structure of the design, the transverse stiffness and direction of the deformation can be angularly regulated, so a single unit can exhibit variable transverse behavior.

Embodiments can be designed without “floating parts” to be realized with stereolithography.

A certain volume can be kept independent from the applied loads to be allocated to nutrients or drugs.

The openings in the extended external surface included in certain embodiments are suitable for modulation with minimal influence on the mechanical behavior, so the release of substances and the biodegradability can be modulated.

Organization of the Units

The improvement in adhesion surface area does not result in an increased scaffold stiffness, and vital space is preserved during deformation independently from the established porosity.

The variation in stiffness can be conferred independently from the given porosity.

Disposing units with different compressibility behavior within the scaffold can generate pressure gradients that result in nutrient and/or fluid flow. For example, motion such as walking results in nutrient motions. The scaffold converts external loads in fluid motion.

Unlike existing scaffolds (in which porosity is used to control the nutrients flow), the invention allows tailoring of the flow independently from the porosity.

Unlike existing devices that convert external loads in fluid motion, embodiments described herein allow localized actions, so the variability in pressure gradients can be associated to specific directionality of the external forces.

Since the scaffold is obtained by combination of structural units described herein, single or multiple units can contain electric motors or can exhibit resonance behavior to enhance the interaction with fluids without influence on porosity.

Algorithm for Patient Specific Design of the Scaffold

While algorithms have been proposed to obtain scaffolds shape to recreate external bone morphology and mechanical properties, embodiments herein comprise an algorithm that accounts for physiologically relevant parameters, such as existing blood vessels, to properly control the flow.

Localized properties are calculated accounting for density, material properties, and vessels.

Existing algorithms target the localized density because associated with mechanical properties. Embodiments herein consider these parameters as independent, so they are independently addressed and optimized in the algorithm.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A structural unit comprising, a central pillar and a plurality of load arms, wherein a load arm of the plurality of load arms comprises a bending arm and a rib, wherein the central pillar and the plurality of load arms are configured to receive at least one force, wherein the bending arm secures the rib to the central pillar, and wherein the rib follows a curved pathway transitioning around an elbow to a return pathway in the direction of the central pillar.

2. The structural unit of claim 1, further comprising a pivot arm, wherein the pivot arm secures the rib to the central pillar.

3. The structural unit of claim 1, wherein the plurality of load arms are integrally formed.

4. The structural unit of claim 1, wherein the curved pathway comprises a constant radius of curvature.

5. The structural unit of claim 1, wherein the curved pathway comprises a variable radius of curvature.

6. The structural unit of claim 1, wherein an axial line and a transverse plane orient the structural unit, wherein the axial line is collinear with a first axis of the central pillar.

7. The structural unit of claim 6, wherein the transverse plane is perpendicular to the axial line.

8. The structural unit of claim 7, wherein the plurality of load arms are oriented about 10°, about 20°, about 30°, about 40°, about 50°, about 60°, about 70°, about 80°, about 90° relative to the transverse plane.

9. The structural unit of claim 7, wherein the plurality of load arms comprise a second axis collinear with the axial line.

10. The structural unit of claim 9, wherein the plurality of load arms are radially spaced about the second axis.

11. The structural unit of claim 7, wherein the curved pathway is concave downward relative to the transverse plane.

12. The structural unit of claim 7, wherein the curved pathway is concave upward relative to the transverse plane.

13. The structural unit of claim 7, wherein the at least one force comprises a vector collinear with the axial line.

14. The structural unit of 13, wherein the force comprises contact pressure, non-contact pressure, or any combination thereof.

15. The structural unit of claim 7, wherein the at least one force comprises a vector oblique to the axial line.

16. The structural unit of claim 7, the receiving the at least one force including causing a deformation of the structural unit along the transverse plane.

17. The structural unit of claim 16, wherein a plurality of cross sectional planes cross section the structural unit, wherein the plurality of cross sectional planes are parallel to the transverse plane.

18. The structural unit of claim 17, wherein each cross section comprises a cross sectional radius.

19. The structural unit of claim 18, wherein the deformation comprises at least one of a contraction, expansion, and stasis of cross sectional radii.

20. The structural unit of claim 19, wherein characteristics of the at least one of a contraction, expansion, and stasis of cross sectional radii depend upon at least one parameter of the structural unit.

21. The structural unit of claim 20, wherein the at least one parameter comprises radial spacing between ribs of the plurality of ribs.

22. The structural unit of claim 20, wherein the at least one parameter comprises variation in thickness of the ribs, variation in the number of ribs, variation in angular width, or any combination thereof.

23. The structural unit of claim 20, wherein the at least one parameter comprises angular width of the plurality of ribs.

24. The structural unit of claim 20, wherein the at least one parameter comprises length of the at least one bending arm.

25. The structural unit of claim 20, wherein the at least one parameter comprises length of the at least one pivot arm.

26. The structural unit of claim 20, wherein the at least one parameter comprises positioning of the at least one pivot arm.

27. The structural unit of claim 7, wherein the transverse plane separates a first set of load arms in a first region and a second set of load arms in a second region, wherein the at least one parameter comprises at least one gap between pivot arms in the first region and pivot arms in the second region, wherein the plurality of load arms comprise the first set of load arms and the second set of load arms.

28. The structural unit of claim 1, wherein the structural unit comprises a sphere, a conical shape, an oval shape, and a cubical shape.

29. The structural unit of claim 28, wherein the sphere comprises a diameter of approximately 50 μm to 2000 μm.

30. The structural unit of claim 1, wherein the structural unit comprises a symmetric shape or an asymmetric shape.

31. The structural unit of claim 1, wherein the structural unit comprises a biocompatible material.

32. The structural unit of claim 31, wherein the biocompatible material comprises a photoresist polymer.

33. The structural unit of claim 1, comprising an external extended surface surrounding the structural unit.

34. The structural unit of claim 33, wherein the external extended surface provides an inner region.

35. The structural unit of claim 34, wherein the inner region comprises a fluid.

36. The structural unit of claim 35, wherein the fluid comprises.

37. The structural unit of claim 34, wherein the external extended surface is fully sealed.

38. The structural unit of claim 34, wherein the external extended surface comprises at least one opening, wherein the at least one opening one or more of releases medications, establishes an equilibrium between external and internal pressures, supplies substances including nutrients, filters a stream surrounding the structural unit, and fit units together.

39. The structural unit of claim 1, comprising a superior portion and an inferior portion.

40. The structural unit of claim 39, wherein the superior portion and the inferior portion are flat.

41. The structural unit of claim 39, wherein the superior portion and the inferior portion are radially extended.

42. The structural unit of claim 1, wherein connecting elements connect the plurality of load arms to the central pillar.

43. A composition comprising two or more structural unites of claim 1 and a pharmaceutically acceptable carrier.

44. A bioscaffold of at least two structural units of any of claims 1-42, wherein the at least two structural units are one or more of indirectly connected using connecting elements and directly connected.

45. The bioscaffold of claim 44, wherein the bioscaffold comprises a sheet, a stack, a spiral, or a linear bioscaffold.

46. The bioscaffold of claim 44, wherein compositions of the at least two structural units are at least one of identical, similar, and different.

47. The bioscaffold of claim 44, wherein at least one of the at least two structural units comprises an electric motor.

48. The bioscaffold of claim 44, wherein at least one of the at least two structural units reacts to external magnetic fields.

49. The bioscaffold of claim 44, wherein the bioscaffold at least one of regulates fluid flow through the bioscaffold, regulates nutrient flow through the bioscaffold, provides mechanical stimuli, and promotes proliferation and survival of cells.

50. The bioscaffold of claim 44, wherein the bioscaffold comprises viable cells.

51. The bioscaffold of claim 50, wherein the cells comprise exogenous cells, autologous cells, and allogenic cells.

52. The bioscaffold of claim 50, wherein the viable cells comprise one or more of osteoblasts, osteaoclasts, lining cells, stromal cells, fibroblasts, endothelial cells, progenitor cells, stem cells, organ-specific cells, tissue-specific cells, keratinocytes, melanocytes, and a nerve cell.

53. A method of regenerating tissue in a subject, the method comprising obtaining a bioscaffold of claim 111 and implanting the bioscaffold to a site on the subject, whereby the bioscaffold is populated with viable cells, thereby regenerating a tissue in a subject.

54. The method of claim 53, wherein the tissue comprises bone.

55. The method of claim 53, wherein the bioscaffold is provided on a screw, prosthesis or implant.
